(19) United States
(12) Patent Application Publication
(10) Pub. No.: US 2007/0185469 A1
(43) Pub. Date: Aug. 9, 2007

(54) Napkin with an adhesive tab

(76) Inventor: John G. Green, Birmingham, AL (US)

Correspondence Address: SUNG I. OH, PROFESSIONAL LAW CORPORATION, 710 QUAIL VALLEY LANE, WEST COVINA, CA, 91791, US

(21) Appl. No.: 11/348,383

(22) Filed: Feb. 6, 2006

(51) Int. Cl. A61F 13/15

(52) U.S. Cl. 604/387; 2/48; 24/7

(57) ABSTRACT

A napkin that is worn for dining includes a strip of adhesive which will releasably attach the napkin to the clothes of a wearer and a release layer for covering the strip of adhesive. The adhesive strip is located on one corner of the napkin and the release layer is located on a corner opposite to the adhesive strip. When the napkin is folded for storage, the release layer covers the adhesive strip, and when the napkin is unfolded for use, the release layer is pulled off of the adhesive strip thereby automatically exposing the adhesive strip for releasable attachment to the clothes of the user. The adhesive strip is covered when the napkin is folded so it will not interfere with the napkin and will not be degraded by contact with other items during storage of the napkin.

### TECHNICAL FIELD OF THE INVENTION

The present invention relates to the general art of dinner napkins, and to the particular field of accessories for dinner napkins.

### BACKGROUND OF THE INVENTION

Cloth and paper napkins have been long in use to protect a diner from food and drink spills. These napkins are usually tucked under the chin or laid on the lap of the user. One problem associated with such napkins and their use is that they are not easily kept in place. They move or inadvertently fall to the floor often unnoticed by the user, and so offer no protection. The hapless diner is left to scramble under the table to retrieve the napkin, which may be soiled, or to procure another napkin. Although these napkins may be pinned, as by safety pins or the like, to the user, such pinning is inconvenient and is rarely used for others than infants.

Fine restaurant provide high quality cloth napkins, which suffer the above limitations, as they are not attachable to the user (not considering tucking it under the chin or inside a shirt). These cloth napkins are economical since they can be washed and reused substantially eliminating the cost of the napkin. However cloth napkins are rarely used outside of restaurants and/or formal dinners since they offer little protection but entail overhead of washing and folding.

The problem of keeping a table napkin in place is especially acute for airline passengers or those in crowded restaurants because of the difficulty of retrieving the napkin within the crowded confines between seats. Moreover, some diners often prefer to have the table napkin extend above the lap to protect against spills. In this regard the art has disclosures of anchors that are used to attach the table napkins above the lap. Such anchors generally a button hole to allow the user to attach the napkin to his shirt or vest buttons. The button hole napkins are generally of no assistance to women whose apparel may lack the requisite buttons in front. Also providing a button hole is generally impractical for paper napkins, which are more likely to slip and slide than cloth napkins.

While the art has examples of means for fixing a dinner napkin to a user, these means have several drawbacks. For example, some of the fixing means are cumbersome to use and require extra steps, such as the removal of a release layer, to activate the attaching means. While this may not appear to be too vexatious, dinner napkins are generally considered elements that are to be as easily deployed as possible and any additional steps required to properly place a dinner napkin on the lap of a user may be considered an inconvenience.

Therefore, there is a need for a dinner napkin that has means for fixing the napkin to the lap of a user which means is easy and expeditious to use yet will still be effective.

### SUMMARY OF THE INVENTION

The above-discussed disadvantages of the prior art are overcome by a dinner napkin that has adhesive on one corner thereof and a release liner fixed to an opposite corner thereof in a position to cover the adhesive when the napkin is folded, but to be removed from the adhesive covering location when the napkin is unfolded for use.

Using the napkin embodying the present invention will permit the napkin to have a user adhering feature that is efficiently stored but which is automatically activated when the napkin is unfolded for use. This will allow the napkin to have the user-adhering features deemed desirable but which will avoid the disadvantages associated with extra steps required to deploy the user-adhering feature.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWING FIGURES

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a perspective view of a napkin embodying the present invention.

FIG. 2 is an example of a strip of low tack adhesive that is located on one corner of the napkin.

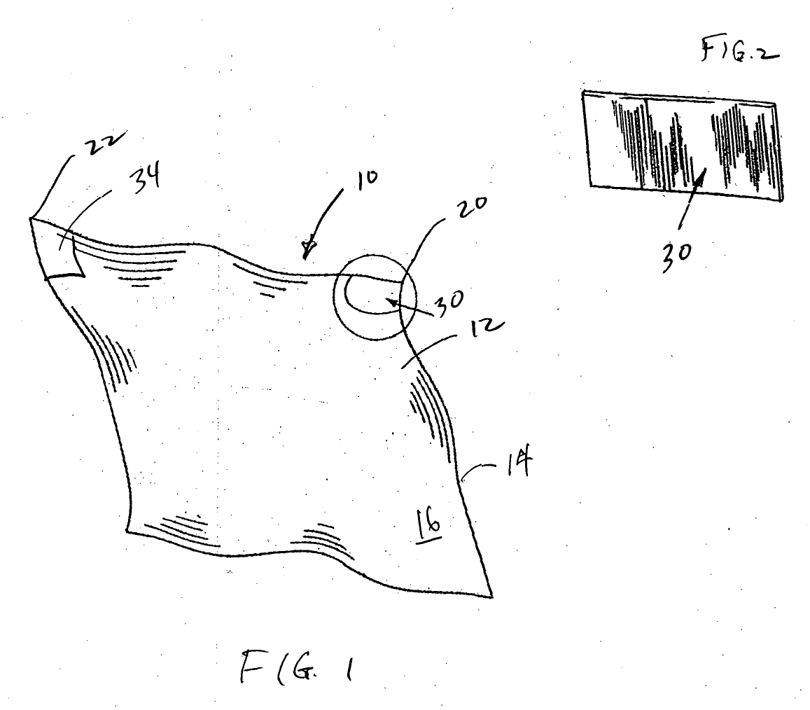

### DETAILED DESCRIPTION OF THE INVENTION

Referring to the figures, it can be understood that the present invention is embodied in a napkin 10 that can be a paper napkin or a cloth napkin and is used to cover a person's clothing while the person is eating. Napkin 10 comprises body 12 which can be polygonal and is generally folded during storage and which is unfolded for use as is will known.

Body 12 has a first surface 14 that is a top surface when the body is in use on a user's lap, a second surface 16 which is a bottom surface when the napkin is in use on a user's lap. Bottom surface 16 contacts the user's clothes when body 12 is in use. Body 12 further includes a first corner 20 and a second corner 22 which is located opposite to the first corner to be spaced apart therefrom.

A strip 30 of adhesive is located on second surface 16 adjacent to first corner 20 and is of the type that will releasably adhere to a user's clothing without damaging that clothing. Those skilled in the art will understand what type of adhesive will be best based on the teaching of the present invention. Accordingly, a detailed discussion of the adhesive will not be presented.

A layer 34 of release liner is fixed to second surface 16 adjacent to second corner 22 to cover the strip of adhesive when the second corner of the body is folded on top of the strip of adhesive and to uncover the strip of adhesive when the second corner is moved away from the first corner. It can be understood from the teaching of the foregoing disclosure that the release liner and the adhesive can be on opposite surfaces if the napkin is to be folded in a particular manner. The only requirement is that the release liner be fixed to the body of the napkin and be located to releasably cover the strip of adhesive when the napkin is in a folded condition.

While various embodiments of the invention have been described, it will be apparent to those of ordinary skill in the art that many more embodiments and implementations are possible within the scope of this invention. Accordingly, the invention is not to be restricted except in light of the attached claims and their equivalents.

1. A napkin consisting of:
A) body which has
(1) a first surface that is a top surface when the body is in use on a user's lap,
(2) a second surface which is a bottom surface when the napkin is in use on a user's lap, the bottom surface contacting the user's clothes when the body is in use,
(3) a first corner, and
(4) a second corner which is located opposite to the first corner;
B) a strip of adhesive located on the second surface adjacent to the first corner; and
C) a layer of release liner fixed to the second surface adjacent to the second corner to cover the strip of adhesive when the second corner of the body is folded on top of the strip of adhesive and to uncover the strip of adhesive when the second corner is moved away from the first corner.

2. A napkin consisting of:
A) a body having a bottom surface when the napkin is in use on a user's lap;
B) a strip of adhesive on the bottom surface of the body; and
C) a release liner fixed to the bottom surface of the body at a location spaced apart from the strip of adhesive and located to releasably cover the strip of adhesive when the body is folded and to be pulled off the strip of adhesive when the body is unfolded.
